(19) United States
(12) Patent Application Publication
(10) Pub. No.: US 2024/0352390 A1
(43) Pub. Date: Oct. 24, 2024

(54) SPIRAL WOUND CELL CULTURE VESSEL AND CELL CULTURE SYSTEM USING SAME

(71) Applicant: CORESTEMCHEMON INC., Seongnam-si, Gyeonggi-do (KR)

(72) Inventors: Kyung Suk KIM, Seoul (KR); Seok Kee HONG, Seoul (KR); Tae Yong LEE, Yongin-si Gyeonggi-do (KR); Si Eun YANG, Guri-si Gyeonggi-do (KR); Min Sung KIM, Seoul (KR); Chang Jae SHIM, Seoul (KR)

(21) Appl. No.: 18/685,764

(22) PCT Filed: Aug. 23, 2021
(86) PCT No.: PCT/KR2021/011213

§ 371 Date: Feb. 22, 2024

(51) Int. Cl. C12M 1/12; C12M 1/00; C12M 3/00

(52) U.S. Cl. CPC C12M 25/00; C12M 23/38; C12M 23/46

(57) ABSTRACT

Provided are a spiral type cell culture vessel and a cell culture system using same. The present invention comprises: a housing having a certain length and having respective ends which are open; first and second caps respectively assembled to one end and the other end of the housing; and at least one spiral type culture support which is disposed in an internal space of the housing and in which a cell culture is performed. The spiral type culture support comprises at least one culture sheet of which one edge is in contact with and is fixed to a support shaft having a certain length, the culture sheet is spirally wound around the support shaft, and a culture medium passes in one direction due to a gap formed between the spirally wound culture sheet and the inner side thereof, and thus, a cell culture space is formed in which the cell culture is performed.

### TECHNICAL FIELD

The present invention relates to a spiral type cell culture vessel and a cell culture system using same, and more particularly, to a spiral type cell culture vessel capable of culturing and harvesting a large amount of cell through a single process and a cell culture system using same.

### BACKGROUND ART

In general, cell culture is a method of removing a tissue piece from a multicellular organism and then supplying nutrients to the removed tissue piece in a vessel to culture or proliferate cells.

The cell culture method includes a cover-glass method, a flask method, and a rotary tube method. In general, an effective method for promoting growth of a cultured tissue includes endosperm, leukocyte, or spleen extracts. However, its essential materials are not clearly elucidated yet. In recent years, antibiotics or eagle culture medium mixed with vitamins and amino acids is being used.

Tissue culture may culture a single cell into a cell population, a small organ, or a plant tissue.

Culturing of a cell living in a test tube is performed for various purposes including recovery of additional by-products of cell metabolism, production of viral vaccines, intentional cultivation of cells to create artificial organs, production of pharmaceuticals through genetic manipulation of animal cells, or breeding through plant cell fusion.

In general, culturing of animal cells requires a culture medium containing nutrients such as amino acids, sugars, inorganic salts, and vitamins, and their culture conditions are complicated. The plant cells have high viability due to their photosynthesis capabilities compared with animal cells, and thus it is easy to culture them but their proliferation rate is slow.

In recent years, a culture method according to characteristics of cells is spotlighted for effective cell culture. As a result, the culture method suitable for the characteristics of cells is researched, developed, and widely used for hybridomas and embryonic stem cells. However, a method for mass culture of adherent cells such as fibroblastoids or epithelial-like cells is not developed yet, and thus the culture of the adherent cells shows a low culture yield and has difficulty in culturing for a long time.

Also, the cell culture requires a certain space in which cells are cultured, a culture medium that supplies nutrients to the cells, and a culture storage space to store the cells. In particular, a culture medium gas and stimulating materials are injected into the culture medium storage space and then required to be replaced at appropriate periods after being used to keep the cell tissue in a fresh condition.

(Patent Document 1) KR10-2019-0010709 A

(Patent Document 2) KR10-1367870 B1

The patent documents 1 and 2 have an advantage of promoting diffusion of nutrients into cells and collection of cell wastes and objects while providing a large surface area in a hollow fiber and proliferating cells with high density compared to other cell culture systems.

However, a cell culture device using the hollow fiber is insufficient in transmission of active oxygen in the cell culture space, is applied for small-scale laboratory research purposes of less than a few hundred liters, and is not suitable for industrial-scale mass production. Thus, the hollow fiber cell culture device has a limitation in culture size, which causes a problem in consideration of a current large-scale industrial level and a low efficiency of separating and collecting cells cultured on a hollow fiber membrane.

### DISCLOSURE OF THE INVENTION

### Technical Problem

The present invention provides a spiral type cell culture vessel capable of easily separating and collecting cells cultured in a culture space to increase a harvest rate of cultured cells, and a cell culture system using same.

The present invention also provides a spiral type cell culture vessel capable of simply increasing or decreasing a space in which cells are cultured by a winding amount of a base material wound in a spiral shape, culturing a large amount of cells, and culturing cells with high quality under an optimized condition, and a cell culture system using same.

Technical objects to be solved by the present invention are not limited to the aforementioned technical objects and unmentioned technical objects will be clearly understood by a person skilled in the art to which the present invention belongs.

### Technical Solution

Embodiments of the present invention provide a spiral type cell culture vessel including: a housing having a certain length with opened both ends; first and second caps respectively assembled to one end and the other end of the housing; and at least one spiral type culture support disposed in an inner space of the housing to perform cell culture, wherein the spiral type culture support includes at least one culture sheet having one edge that is in contact with and fixed to a support shaft having a certain length, the culture sheet is wound in a spiral shape around the support shaft, a gap formed between an inner side of the spirally wound culture sheet and the culture sheet allows a culture medium to pass in one direction, thereby forming a cell culture space in which the cell culture is performed.

Here, the spiral type culture support may include a laminated culture sheet obtained by vertically laminating two sheets of a first culture sheet having one edge fixed to the support shaft having a certain length and a second culture sheet in which peaks and valleys are repeatedly formed from one end fixed to one edge of one surface of the first culture sheet to the other end fixed to the other edge of one surface of the first culture sheet, and the laminated culture sheet that is spirally wound around the support shaft may form a gap between the first culture sheet and the second culture sheet by peaks and valleys of the culture sheet to allow the culture medium to pass in one direction, thereby forming a cell culture space in which the cell culture is performed.

Here, the spiral type culture support may be formed such that the second culture sheet having overall uniform heights or pitches of the valleys and peaks is disposed between an outer surface and an inner surface, which face each other, of the first culture sheet wound around the support shaft at least two times.

Here, the spiral type culture support may be formed such that the second culture sheet having overall uniform heights or pitches of the valleys and peaks is disposed between an outer surface and an inner surface, which face each other, of the first culture sheet wound around the support shaft at least two times, so that the heights or the pitches of the valleys and peaks are gradually increased in a spiral direction.

Here, the second culture sheet may have one end that is fixed to one edge of one surface of the first culture sheet by an adhesive and the other end that is fixed to the other edge of the one surface of the first culture sheet by the adhesive.

Here, a lower portion of each of the valleys of the second culture sheet, which is in contact with one surface of the first culture sheet, may be fixed by an adhesive.

Here, the spiral type culture support may include a single culture sheet made of a third culture sheet which has one edge fixed to the support shaft having a certain length and in which a plurality of linear protrusions are spaced a certain distance from each other on one surface to repeatedly form peaks and valleys, and the single culture sheet that is wound in a cylinder shape around the support shaft may form a gap between the third culture sheets by the linear protrusions that form the peaks and valleys to allow the culture medium to pass in one direction, thereby forming a cell culture space in which the cell culture is performed.

Here, the spiral type culture support may be formed such that the linear protrusions having overall uniform heights or pitches is disposed between an outer surface and an inner surface, which face each other, of the third culture sheet wound around the support shaft at least two times.

Here, the spiral type culture support may be formed such that the linear protrusions each having a height or a pitch that is gradually increased in a spiral direction is disposed between an outer surface and an inner surface, which face each other, of the third culture sheet wound around the support shaft at least two times.

Here, the spiral type culture support may include a single culture sheet made of a fourth culture sheet which has one edge fixed to the support shaft having a certain length and in which a plurality of protruding embossings are spaced a certain distance from each other on one surface, and the single culture sheet that is wound in a cylinder shape around the support shaft may form a gap between the fourth culture sheets wound by the embossings to allow the culture medium to pass in one direction, thereby forming a cell culture space in which the cell culture is performed. Here, the spiral type culture support may be formed such that the embossings having overall uniform heights or pitches is disposed between an outer surface of the fourth culture sheet and an inner surface of the third culture sheet, which are wound around the support shaft at least two times to face each other.

Here, the spiral type culture support may be formed such that the embossings each having a height or a pitch that is gradually increased in a spiral direction is disposed between an outer surface and an inner surface, which face each other, of the fourth culture sheet wound around the support shaft at least two times.

Here, a first space in which the culture medium is temporarily retained and stored may be formed between the first cap and one end of the corresponding spiral type culture support.

Here, a second space in which the culture medium is temporarily retained and stored may be formed between the second cap and the other end of the corresponding spiral type culture support.

Also, a cell culture system includes: a first actuator configured to operate a first adapter selectively one-to-one corresponding to the first cap or second cap of the housing provided in the spiral type cell culture vessel, so that the first adapter is selectively communicated and connected to a first inflow and outflow hole of the first cap or a second inflow and outflow hole of the second cap; a second actuator configured to operate a second adapter selectively one-to-one corresponding to the first cap or second cap of the housing, so that the second adapter is selectively communicated and connected to the first inflow and outflow hole of the first cap or the second inflow and outflow hole of the second cap; and a pump disposed on a middle portion of a length of a connection line that is communicated and connected between the first adapter and the second adapter to provide the culture medium stored in a culture medium tank toward the spiral type culture support of the housing and then forcefully circulate the culture medium so that the culture medium discharged from the housing through the cell culture space of the spiral type culture support is returned toward the culture medium tank.

Here, the cell culture system may further include an invert actuator configured to provide an external force to invert the housing by 180° through a gripper configured to clamp the housing.

### Advantageous Effects

According to the present invention, the large amount of cells may be cultured through the single process to improve the productivity, reduce the costs, and maintain the uniformity of the quality.

### BRIEF DESCRIPTION OF THE DRAWINGS

FIG. 1 is an overall configuration view illustrating a cell culture system using a spiral type cell culture vessel according to an embodiment of the present invention.

FIG. 2 is a view illustrating the spiral type cell culture vessel according to an embodiment of the present invention.

FIG. 3 is an exploded perspective view illustrating the spiral type cell culture vessel according to an embodiment of the present invention.

FIGS. 4a, 4b, and 4c are configuration views illustrating a first embodiment of a spiral type culture support provided in the spiral type cell culture vessel according to an embodiment of the present invention.

FIGS. 5a, 5b, and 5c are configuration views illustrating a modified example of the first embodiment of the spiral type culture support provided in the spiral type cell culture vessel according to an embodiment of the present invention.

FIGS. 6a, 6b, and 6c are configuration views illustrating a second embodiment of the spiral type culture support provided in the spiral type cell culture vessel according to an embodiment of the present invention.

FIGS. 7a, 7b, and 7c are configuration views illustrating a third embodiment of the spiral type culture support provided in the spiral type cell culture vessel according to an embodiment of the present invention.

FIGS. 8a and 8b are schematic views illustrating an invert operation of the spiral type cell culture vessel in the cell culture system according to an embodiment of the present invention.

### MODE FOR CARRYING OUT THE INVENTION

Hereinafter, preferred embodiments of the present disclosure will be described in detail with reference to the accompanying drawings in such a manner that the technical idea of the present disclosure may easily be carried out by a person skilled in the art to which the invention pertains. Moreover, detailed descriptions related to well-known functions or configurations will be ruled out in order not to unnecessarily obscure subject matters of the present invention.

Also, portions having similar function and effect may be expressed with the same or similar reference symbol throughout.

In addition, in this disclosure below, when one part (or element, device, etc.) is referred to as being ‘connected’ to another part (or element, device, etc.), it should be understood that the former can be ‘directly connected’ to the latter, or ‘indirectly connected’ to the latter via an intervening part (or element, device, etc.). Furthermore, when it is described that one comprises (or includes or has) some elements, it should be understood that it may comprise (or include or has) only those elements, or it may comprise (or include or have) other elements as well as those elements if there is no specific limitation.

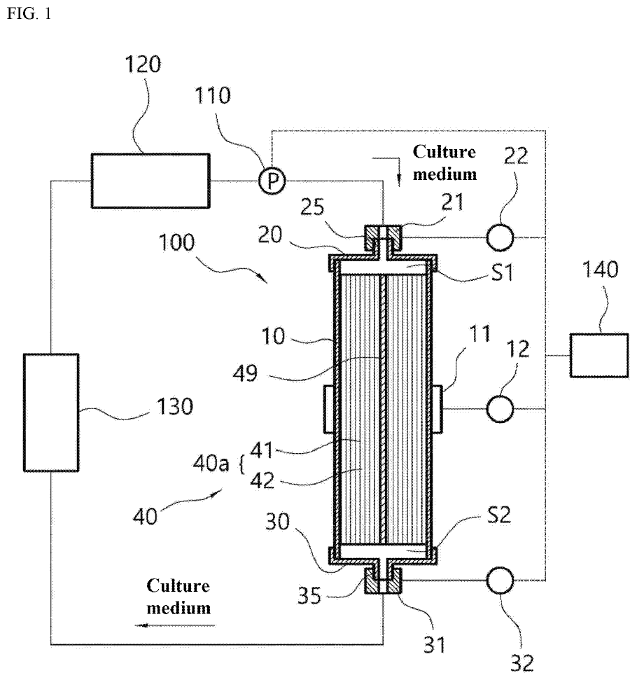

As illustrated in FIGS. 1, 2, and 3, a spiral type cell culture vessel (100) according to a preferred embodiment of the present invention may include a housing (10), first and second caps (each 20 and 30), and a spiral type culture support (40) to culture a cell contained in a culture medium by attaching the cell to the vessel while the culture medium passes in one direction, culture a cell attached in advance, or separate and harvest a cultured cell.

The culture medium may be prepared as a solution containing adherent cells in such a manner that all microorganisms are removed through a sterilization treatment by applying heat under a certain pressure for a specific time in an autoclave, and then substances necessary for growth of bacteria, protozoa, algae, and fungi are added.

The housing (10) may be a hollow cylinder which has a certain length and opened both ends and in which the spiral type culture support is disposed.

An outer diameter and size of the housing (10) may be designed and changed to various sizes to match an outer diameter and length of the culture support (40) which is disposed in an inner space and in which cell culture is performed.

The first cap (20) is detachably assembled to an opened one end of the housing (10) to seal opened one end of the housing and, at the same time, includes a first inflow and outflow hole (25) communicating with a first adapter (21) that will be described later to form an inflow and outflow passage of a circulating culture medium.

The second cap (30) is detachably assembled to an opened one end of the housing (10) in the same manner as the first cap to seal opened the other end of the housing and, at the same time, includes a second inflow and outflow hole (35) communicating with a second adapter (31) that will be described later to form an inflow and outflow passage of the circulating culture medium.

Here, the first and second caps (each 20 and 30) are detachably coupled to the opened both ends of the housing (10) in a press-fitting method, respectively, but the embodiment of the present invention is not limited thereto. For example, the first and second caps (each 20 and 30) may include a female thread that is screwed with a male thread formed on an outer surface of both the ends of the housing and be detachably coupled to opened both the ends of the housing (10) in a screw fastening method.

Here, a coupling portion between the first and second caps and both the ends of the housing may be sealed by a sealing member such as an O-ring to prevent the culture medium flowing in and out of the housing through the first and second inflow and outflow hole from being leaked.

The spiral type culture support (40), in which cell culture is substantially performed, in the housing (10) may be easily and quickly removed and withdrawn or replaced with a new one by selectively separating the first cap (20) or the second cap (30).

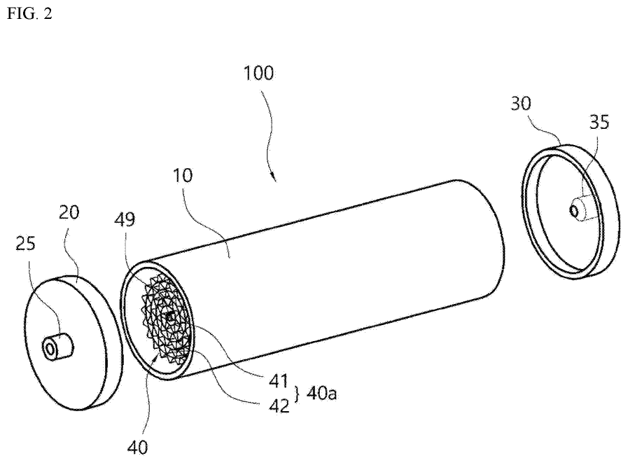

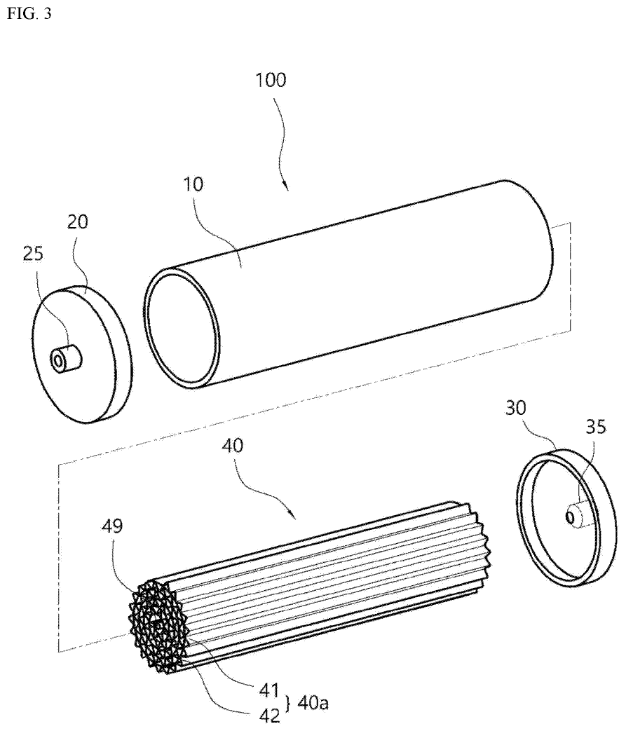

As illustrated in FIGS. 2 and 3, at least one spiral type culture support (40) is disposed in the inner space of the housing (10), and culture is performed by attaching cells contained in the culture medium to the support or cells attached to the support in advance is cultured.

The spiral type culture support (40) may include at least one culture sheet having one edge that is in contact with and fixed to an outer surface of a support shaft (49) having a certain length. As the culture sheet is wound in a spiral shape using the support shaft as the center, a gap may be formed between an inner side of the cylindrically wound culture sheet and the culture sheet, and this gap may allow the culture medium to pass in one direction, thereby forming a cell culture space in which cell culture is performed.

That is, the spiral type culture support (40) may include a laminated culture sheet (40a) manufactured by vertically laminating two sheets of a first culture sheet (41) and a second culture sheet (42). The first culture sheet (41) having one edge that is fixed to the support shaft (49) having a certain length, and the second culture sheet (42) in which peaks and valleys are repeatedly formed from one end fixed to one edge of one surface of the first culture sheet (41) to the other end fixed to the other edge of one surface of the first culture sheet (41).

The first culture sheet (41) may be made of a culture sheet material having an approximately rectangular plate shape, and the second culture sheet (42) may be made of a corrugated culture sheet material in which peaks and valleys are continuously formed from one edge to the other edge.

As the spiral type culture support is configured by winding the laminated culture sheet (40a) obtained by vertically laminating the first culture sheet (41) and the second culture sheet (42) in which the peaks and valleys are repeatedly formed in the spiral shape around the support shaft (49), the peaks and valleys of the second culture sheet are wound to contact inner and outer surfaces of the facing first culture sheet (41) to form a gap having a certain size between the first culture sheet (41) and the second culture sheet (42).

Here, the gap formed between the first culture sheet (41) and the second culture sheet (42) may be regulated and controlled by the peaks and valleys.

Accordingly, as the gap is formed between the first culture sheet (41) and the second culture sheet (42) by the peaks and valleys of the second culture sheet (42) in contact with the first culture sheet, a cell culture space through which the culture medium provided into the housing passes in one direction may be formed by the gap, and a culture process may be performed while cells contained in the culture medium are attached to surfaces of the first and second culture sheets forming the cell culture space, or a culture process may be performed on the cells attached to the surfaces of the first and second culture sheets.

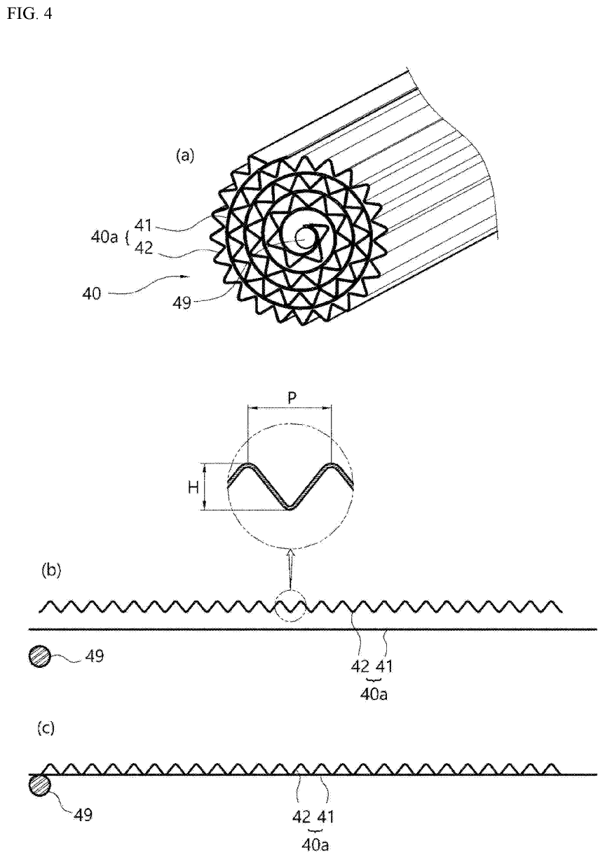

As illustrated in FIGS. 4a, 4b, and 4c, the peaks and valleys repeatedly formed on the second culture sheet (42) are formed between adjacent peaks and between adjacent valleys with an overall uniform pitch (P) and an overall uniform height (H).

In this case, the spiral type culture support (40) obtained by winding the laminated culture sheet (40a) in which the first and second culture sheets (each 41 and 42) are laminated around the support shaft (49) in a cylindrical shape may have a structure in which the second culture sheet (42) having the overall uniform height (H) of the peaks and valleys is disposed between an outer surface and an inner surface, which face each other, of the first culture sheet (41) wound around the support shaft (49) at least two times, to secure and constantly maintain a gap between the outer surface of the first culture sheet (41) disposed at an inner side and the inner surface of the first culture sheet (41) disposed at an outer side. Thus, a flow velocity of the culture medium passing in one direction through the gap may be constantly maintained, and a process of culturing the cells attached to the second culture sheet may be performed.

Here, although it is illustrated and described that the pitch (P) between the valleys or the pitch (P) between the peaks formed in the second culture sheet (42) has the same overall size, the embodiment of the present invention is not limited thereto. For example, the pitches may have different sizes.

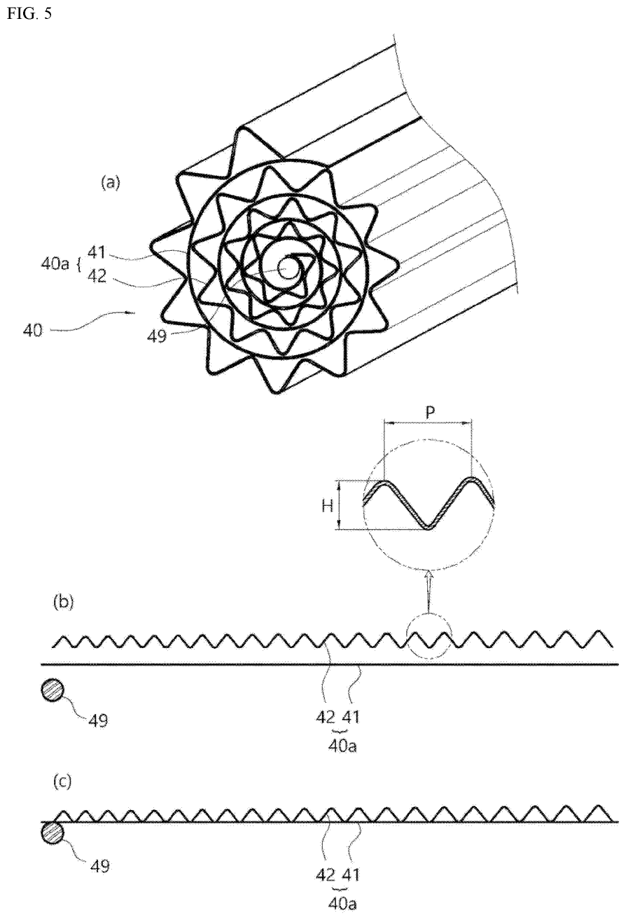

As illustrated in FIGS. 5a, 5b, and 5c, the second culture sheet (42) may have the overall uniform pitch (P) between adjacent peaks and between adjacent valleys, so that the height (H) of the adjacent peaks is gradually increased in a direction away from the support shaft (49).

In this case, the spiral type culture support (40) obtained by winding the laminated culture sheet (40a) in which the first and second culture sheets (each 41 and 42) are laminated around the support shaft (49) in a cylindrical shape may have a structure in which the second culture sheet (42) having the height (H) of the peaks and valleys, which is gradually increased in a spiral direction, is disposed between an outer surface and an inner surface, which face each other, of the first culture sheet (41) wound around the support shaft (49) at least two times, so that a gap between the outer surface of the first culture sheet (41) disposed at an inner side and the inner surface of the first culture sheet (41) disposed at an outer side is gradually increased in the spiral direction. Thus, since the gap between the outer surface of the first culture sheet (41) disposed at the inner side and the inner surface of the first culture sheet (41) disposed at the outer side may be gradually increased in the spiral direction, the cell culture space of the spiral type culture support may be increased in the direction away from the support shaft (49) to increase an attached amount and harvest amount of the cells.

Here, the pitch between the valleys or between the peaks formed in the second culture sheet (42) may have the same size or a size that is gradually increased in the direction away from the support shaft (49), thereby increasing the cell culture space.

The second culture sheet (42), in which the peaks and valleys are repeatedly formed, may form the laminated culture sheet (40a) having one end fixed to one edge of one surface of the first culture sheet (41) through an adhesive and the other end that is in contact with and fixed to the other edge of the one surface of the first culture sheet (41) through the adhesive so that the second culture sheet (42) is laminated and fixed to the one surface of the first culture sheet (41) or the laminated culture sheet (40a) in which a lower portion of each of the valleys in contact with one surface of the first culture sheet (41) is fixed through the adhesive so that the second culture sheet is laminated and fixed to one surface of the first culture sheet (41).

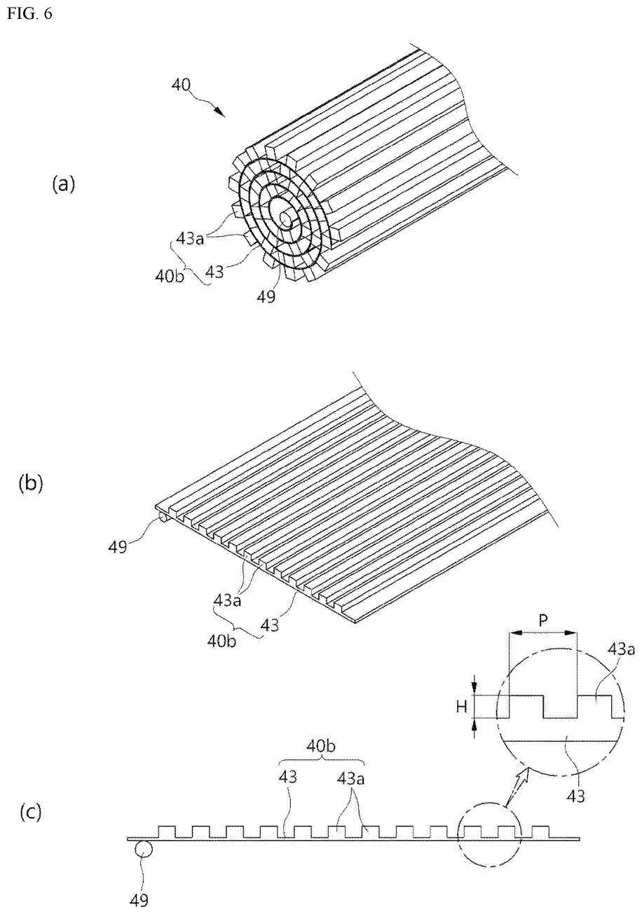

As illustrated in FIGS. 6a, 6b, and 6c, the spiral type culture support (40) may include a single culture sheet (40b) made of a third culture sheet (43) having one edge fixed to the support shaft (49) having a certain length and one surface on which a plurality of linear protrusions (43a) are spaced a certain distance from each other to repeatedly form peaks and valleys.

The third culture sheet (43) may be made of a culture sheet material having an approximately rectangular plate shape so that the linear protrusions (43a) are provided continuously from one edge to the other edge.

The single culture sheet (40b) made of the third culture sheet (43) in which the plurality of linear protrusions (43a) protrude from one surface constitutes the spiral wound support (40) wound in the spiral shape around the support shaft (49) to form a gap having a certain size between the third culture sheets (43) while being brought into contact with the other surface of the third culture sheet (43) facing the linear protrusions (43a) of the third culture sheet.

Here, the gap formed between the wound third culture sheets (43) may be regulated and controlled by the linear protrusions (43a).

Accordingly, as the gap is formed between the wound third culture sheet by the linear protrusions, a cell culture space through which the culture medium provided into the housing passes in one direction may be formed by the gap, and a culture process may be performed while the cells contained in the culture medium are attached to a surface of the third culture sheet forming the cell culture space, or a culture process may be performed on the cells attached to the surfaces of the third culture sheet.

As illustrated in FIGS. 6a, 6b, and 6c, the linear protrusions (43a) that repeatedly form the peaks and valleys on the second culture sheet (43) may have an overall uniform pitch (P) formed between adjacent linear protrusions (43a) and an overall uniform height (H) of the adjacent linear protrusions (43a).

In this case, the spiral type culture support (40) obtained by winding the single culture sheet (40b) made of one third culture sheet (43) around the support shaft (49) in a cylindrical shape may have a structure in which the linear protrusions (43a) are disposed between an outer surface and an inner surface, which face each other, of the third culture sheet (43) wound around the support shaft (49) at least two times to secure and constantly maintain a gap between an outer surface of the third culture sheet (43) disposed at an inner side and an inner surface of the third culture sheet (43) disposed at an outer side. Thus, a flow velocity of the culture medium passing in one direction through the gap may be constantly maintained, and a process of culturing the cells attached to the third culture sheet may be performed.

Here, the pitches between the plurality of linear protrusions (43a) protruding from one surface of the third culture sheet (43) may have an overall same size or different sizes.

The third culture sheet (43) may have a structure having an overall uniform pitch (P) between adjacent linear protrusions and the height (H) of the linear protrusions (43a), which is gradually increased in the direction away from the support shaft (49).

In this case, the spiral type culture support (40) obtained by winding the single culture sheet (40b) made of the third culture sheets (43) around the support shaft (49) in a cylindrical shape may have a structure in which the linear protrusions (43a) is disposed between the outer surface and the inner surface of the third culture sheet (43) wound around the support shaft (49) at least two times, so that a gap between the outer surface of the third culture sheet (43) disposed at an inner side and the inner surface of the third culture sheet (43) disposed at an outer side is gradually increased in the spiral direction. Thus, the cell culture space of the spiral type culture support may be increased in the direction away from the support shaft (49) to increase the attached amount and harvest amount of the cells.

Here, the pitches (P) between the plurality of protruding linear protrusions from the third culture sheet (43) or the heights (H) of the linear protrusions may have an overall same size or a size that is gradually increased in the direction away from the support shaft (49), thereby increasing the cell culture space.

Although it is illustrated and described that the plurality of protruding linear protrusions (43a) that are spaced a certain distance from each other to repeatedly form the peaks and valleys in the third culture sheet have an approximately rectangular cross-section, the embodiment of the present invention is not limited thereto. For example, the linear protrusion (43a) may have various cross-sectional shapes such as an arc, a semi-circle, and a polygon depending on a design.

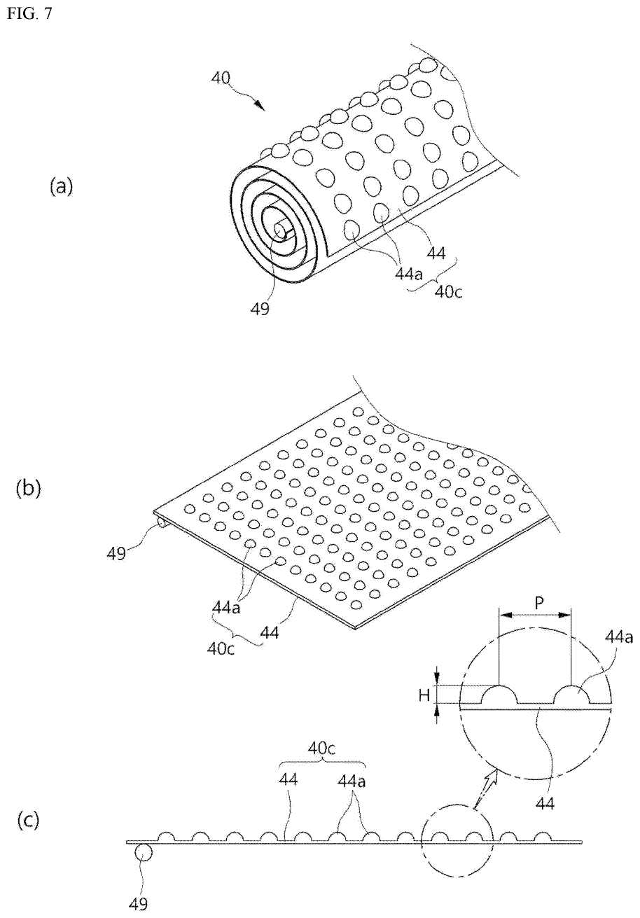

As illustrated in FIGS. 7a, 7b, and 7c, the spiral type culture support (40) may include a single culture sheet (40c) made of a fourth culture sheet (44) having one edge fixed to the support shaft (49) having a certain length and one surface from which a plurality of linear protrusions (44a) protrude.

The fourth culture sheet (44) may be made of the culture sheet material having an approximately rectangular plate shape as with the first, second, and third culture sheets.

Another single culture sheet (40c) made of the fourth culture sheet (44) having one surface from which the plurality of linear protrusions (44a) protrude constitutes the spiral wound support (40) wound in the spiral shape around the support shaft (49) to form a gap having a certain size between the fourth culture sheets (44) while being brought into contact with the other surface of the fourth culture sheet (44) facing the embossings (44a) of the fourth culture sheet.

Here, the gap formed between the wound fourth culture sheets (44) may be regulated and controlled by the embossings (44a).

Accordingly, as the gap is formed between the wound fourth culture sheet by the embossings, a cell culture space through which the culture medium provided into the housing passes in one direction may be formed by the gap, and a culture process may be performed while the cells contained in the culture medium are attached to a surface of the fourth culture sheet forming the cell culture space, or a culture process may be performed on the cells attached to the surface of the fourth culture sheet.

As illustrated in FIGS. 7a, 7b, and 7c, the embossings (44a) that are formed on the fourth culture sheet (44) may have an overall uniform pitch (P) formed between adjacent embossings (44a) and an overall uniform height (H) of the adjacent embossings (44a).

In this case, the spiral type culture support (40) obtained by winding the single culture sheet (40c) made of one fourth culture sheet (44) around the support shaft (49) in an approximately cylindrical shape may have a structure in which the embossings (44a) are disposed between an outer surface and an inner surface, which face each other, of the fourth culture sheet (44) wound around the support shaft (49) at least two times to secure and constantly maintain a gap between an outer surface of the fourth culture sheet (44) disposed at an inner side and an inner surface of the fourth culture sheet (44) disposed at an outer side. Thus, a culture process may be performed on cells attached to the fourth culture sheet while constantly maintaining a flow velocity of the culture medium passing in one direction through the gap.

Here, the pitches between the plurality of embossings (44a) protruding from one surface of the fourth culture sheet (44) may have an overall same size or different sizes.

The fourth culture sheet (44) may have a structure having an overall uniform pitch (P) between adjacent embossings and a height (H) of the embossings (44a), which is gradually increased in the direction away from the support shaft (49).

In this case, the spiral type culture support (40) obtained by winding the single culture sheet (40c) made of one fourth culture sheet (44) around the support shaft (49) in a cylindrical shape may have a structure in which the embossings (44a) are disposed between the outer surface and the inner surface of the fourth culture sheet wound around the support shaft (49) at least two times, so that a gap between the outer surface of the fourth culture sheet disposed at an inner side and the inner surface of the fourth culture sheet disposed at an outer side is gradually increased in the spiral direction. Thus, the cell culture space of the spiral type culture support may be increased in the direction away from the support shaft (49) to increase the attached amount and harvest amount of the cells.

Here, the pitches (P) between the plurality of protruding embossings or the heights (H) of the embossings may have an overall same size or a size that is gradually increased in the direction away from the support shaft (49), thereby increasing the cell culture space.

It is illustrated and described that the plurality of protruding embossings (44a) that are spaced a certain distance from each other in the fourth culture sheet (44) have an approximately semi-circular cross-section, but the embodiment of the present invention is not limited thereto. For example, the embossings may each have various polygonal cross-sectional shapes such as a triangle and a rectangle depending on a design.

On the other hand, one of the first, second, third and fourth culture sheets constituting the spiral type culture support (40) may include a coating layer made of a biocompatible polymer containing at least one selected from the group consisting of PES (Polyethersulfone), PVDF (Polyvinylidene fluoride), PA (Polyamide), PS (polystyrene), PP (Polypropylene), PE (Polyethylene), PS (Polysulfone), PVA (Polyvinylalcohol), and PDMS (poly(dimethylsiloxane), or a biocompatible polymer on a surface of a molded sheet structure.

Here, the biocompatible polymer that is harmless to a human body represents a synthetic or natural polymer material that does not emit harmful substances to the human body even when directly contacts cells or wounds, does not cause side effects such as skin irritation, and has no harmful effects on the human body. The biocompatible polymer may be used without limitation as long as the biocompatible polymer is usable as a dressing material and appropriately selected by a person skilled in the art.

The cells to be cultured and harvested in the spiral type culture support (40) may be skin cells or stem cells, and the skin cells may represent cells that form skin. However, the embodiment of the present invention is not limited to the kinds of cells. For example, the skin cells may be keratinocytes or melanocytes disposed in the epidermis, or fibroblasts, endothelial cells, or hair follicle stem cells disposed in the dermis to perform a biosynthesis of collagen and elastin.

Although the kind of stem cells is not particularly limited, the stem cells may be, e.g., embryonic stem cells, induced pluripotent stem cells, or adult stem cells. The embryonic stem cells may include all embryonic stem cells derived from mammals, e.g., human embryonic stem cells, and the induced pluripotent stem cells may be cells obtained by returning somatic stem cells that have undergone differentiation to a cell stage before the differentiation. Also, the adult stem cells that are derived from adult skin, liver, lung, blood, bone marrow, fat, amniotic membrane, endometrial tissue, cartilage tissue, and umbilical cord blood may be differentiate into all tissues.

On the other hand, a first space (S1) in which the culture medium provided or discharged through the first inflow and outflow hole (25) of the first cap (20) is temporarily retained and stored may be formed between each one end of the spiral type culture support (40) around which the laminated culture sheet (40a) or the single culture sheet (each 40b and 40c) are wound in the cylindrical shape, and the corresponding first cap (20).

A second space (S2) in which the culture medium provided or discharged through the second inflow and outflow hole (35) of the second cap (30) is temporarily retained and stored may be formed between each the other end of the spiral type culture support (40) around which the laminated culture sheet (40a) or the single culture sheets (each 40b and 40c) are wound in the cylindrical shape and the corresponding second cap (30).

Accordingly, when the housing (10) and the spiral type culture support (40) disposed in the housing are installed approximately perpendicular to a virtual horizontal surface so that the first cap (20) of the cell culture vessel (100) is disposed at an upper portion and the second cap (30) is disposed at a lower portion, the culture medium forcibly pumped through the inflow and outflow hole of the first cap (20) may be temporarily retained in the first space and then dropped smoothly and uniformly in a direction of own weight thereof through the cell culture space formed between the first and second culture sheets (each 41 and 42) of the spiral type culture support without increasing internal pressure of the housing (10) more than necessary.

At the same time, the culture medium discharged through a lower end of the spiral type culture support (40) may be temporarily retained in the second space (S2) and then discharged to circulate to the outside of the housing through the second inflow and outflow hole (35) of the second cap (30) without increasing the internal pressure of the housing (10) more than necessary.

As illustrated in FIG. 1, the cell culture system using the cell culture vessel (100) having the above-described configuration may include an invert actuator (12), first and second actuators (each 22 and 32), a pump (110), a culture medium tank (130), and a control unit (140).

The invert actuator (12) provides an external force that inverts the housing (10) by 180° through a gripper (11) that clamps or unclamps an outer surface of the housing (10) of the cell culture vessel (100) disposed approximately perpendicular to the horizontal plane.

Accordingly, the housing (10) in which the spiral type culture support (40) is disposed may be inverted 180° by the invert actuator (12) to invert positions of the first cap and the second cap of the housing, thereby inverting the housing that is vertically disposed.

Here, the inverting of the housing (10) disposed approximately perpendicular to the horizontal plane and the spiral type culture support disposed in the housing is performed in a state in which the first and second adapters (each 21 and 31) connected in communication with the first and second inflow and outflow holes (each 25 and 35) of the first and second caps (each 20 and 30) are separated from each other.

The first actuator (22) provides an external force to ascend the first adapter (21) in a vertical direction, thereby selectively separating the first and second inflow and outflow holes of the first and second caps or descend the first adapter (21) in the vertical direction, thereby selectively being connected in communication with the first and second inflow and outflow holes of the first and second caps, so that the first adapter (21) that selectively one-to-one correspond to the first cap (20) or the second cap (30) of the housing (10) is selectively connected in communication with the first inflow and outflow hole (25) of the first cap (20) or the second inflow and outflow hole (35) of the second cap (30) according to an invert state of the housing.

As with the first actuator, the second actuator 32 provides an external force to ascend the second adapter (31) in the vertical direction, thereby selectively separating the first and second inflow and outflow holes of the first and second caps or descend the second adapter (31) in the vertical direction, thereby selectively being connected in communication with the first and second inflow and outflow holes of the first and second caps, so that the second adapter (31) that selectively one-to-one correspond to the first cap (20) or the second cap (30) of the housing (10) is selectively connected in communication with the first inflow and outflow hole (25) of the first cap (20) or the second inflow and outflow hole (35) of the second cap (30) according to an invert state of the housing.

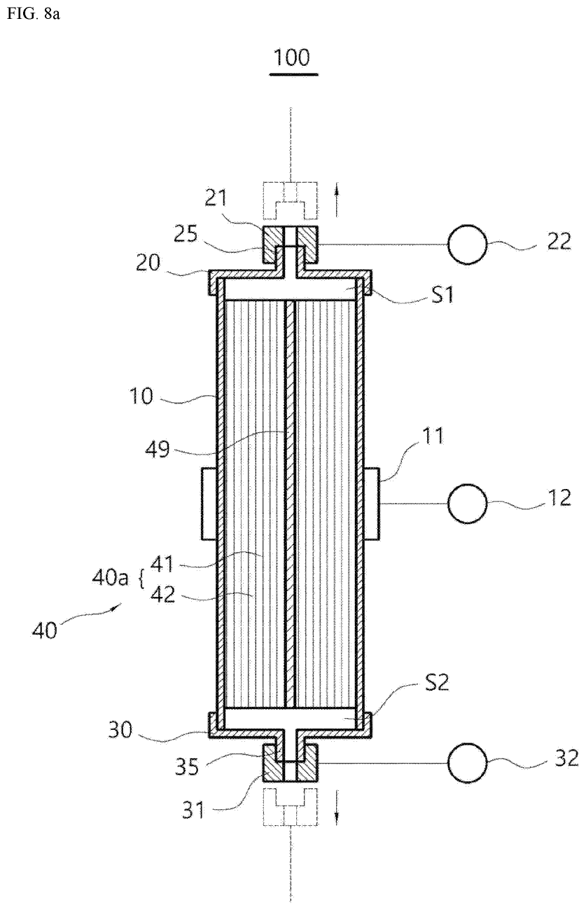

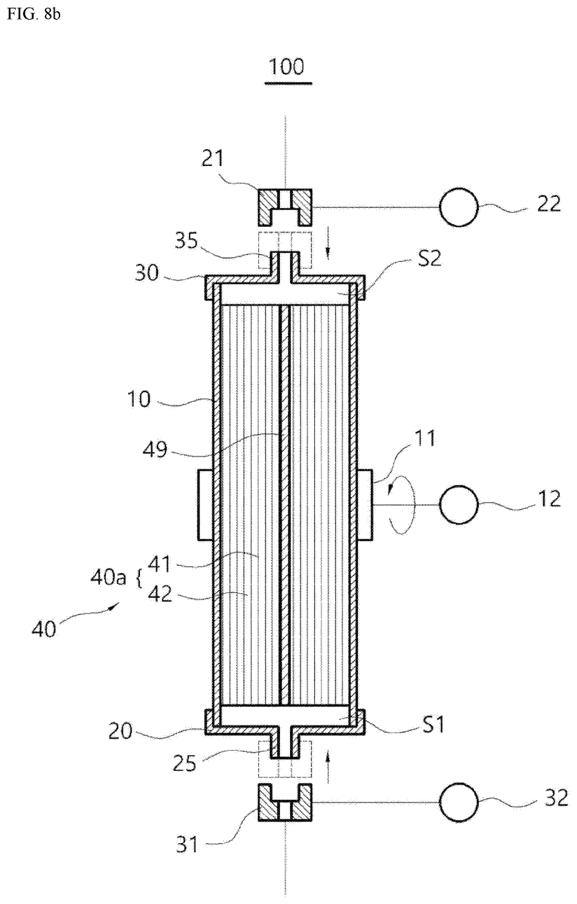

Accordingly, as illustrated in FIGS. 8a and 8b, the housing (10) in which the spiral type culture support (40) is disposed may be inverted 180° by the invert actuator (12) separated from the first and second adapters (each 21 and 31) connected in communication with the corresponding first and second inflow and outflow hole (each 25 and 35) of the first and second caps (each 20 and 30) by the first and second actuators (22) and 32, so that the housing disposed vertically while inverting the vertical positions of the first and second caps and the spiral type culture support disposed in the housing are inverted.

As the cells of the culture medium passing through the cell culture space of the spiral type culture support (40) are attached uniformly to the first and second culture sheets (each 41 and 43) of the laminated culture sheet (40a) or the third and fourth culture sheets (each 43 and 44) of the single culture sheets (40b) and (40c), a culture efficiency and a harvest rate of the cultured cells may be increased.

Here, the cells cultured while being attached to the first, second, third, and fourth culture sheet may be adherent cells contained in the culture medium provided into the housing, but the embodiment of the present invention is not limited thereto. The cells may be adherent cells that is attached in advance to one surface of each of the first, second, third and fourth culture sheets that are unwound on a plane before the laminated culture sheet or single culture sheet constituting the spiral type culture support is wound in the cylindrical shape.

The pump (110) circulates the culture medium discharged to a lower side of the housing while pumping the culture medium into the housing accommodating the spiral type culture support when power is applied.

The pump (110) is disposed in the middle of the length of the connection line that connects and communicates the first adapter (21) and the second adapter (31) and include a culture medium tank (130) in which a certain amount of culture medium pumped by a pumping force of the pump is stored is disposed on the connection line between an inlet of the pump (110) and an outlet of the second adapter (31).

A bubble trap (120) that removes air bubbles contained in the circulating culture medium may be disposed on the connection line between the culture medium tank (130) and the pump (110).

The control unit (140) electrically connected to the pump (110), the invert actuator (12), and the first and second actuators (each 22 and 32) may control: a pumping operation of the pump (110) to pump and supply a certain amount of culture medium into the housing according to a cell culture condition set in advance by the control unit (140); an inversion operation of the invert actuator (12) to invert the housing (10) by 180° according to a previously set inversion time; and an operation of the first and second actuators that allow the first and second adapters (each 21 and 31) to vertically reciprocate.

As carbon dioxide having a certain concentration is continuously supplied to the culture medium tank (130), the culture medium circulating through the pump (110) and the spiral type culture support (40) of the spiral type cell culture vessel through the connection line may maintain a constant pH suitable for cell culture.

Accordingly, the spiral type cell culture system may reuse the culture medium required for cell culture to minimize a use amount of the culture medium, thereby reducing production costs.

Accordingly, the cells attached to the first and second culture sheets of the laminated culture sheet and the third and fourth culture sheets of the single culture sheet, which form the cell culture space of the spiral type culture support, may be cultured smoothly with nutrients supplied from the circulating culture medium, and as an area in which the cells are attached to the second culture sheet in which the valleys and peaks are repeatedly formed, a large amount of cells may be cultured through a single culture process.

Also, when culture of the cells attached to the first and second culture sheets or the third and fourth culture sheets forming the cell culture space of the spiral type culture support (40) is completed, a high-pressure gas is supplied into the housing through a gas supply line (not shown) connected to one of the first and second adapters connected in communication with the first and second caps of the housing, and the culture medium retained in the housing is discharged to the outside by the high-pressure gas through a gas discharge line (not shown) connected to the other of the first and second adapters connected in communication with the first and second caps of the housing.

Here, the gas supply line and gas discharge line are maintained in a closed state by an opening and closing valve during the cell culture process and are selectively opened only when discharging the retained culture medium.

Thereafter, when the culture medium retained in the housing (10) and the cell culture space of the spiral type culture support (40) disposed in the housing is completely removed, a cleaning solution is supplied into the housing (10) through a cleaning water supply line (not shown) branched from the connection line connecting the pump and the first adapter.

Accordingly, the cleaning solution cleans the inside of the housing and the spiral type culture support and then is discharged to the outside through the cleaning water discharge line (not shown) branched from the connection line connecting the culture medium tank (130) and the second adapter.

Finally, when the cleaning process on the cell culture space of the spiral type culture support (40) and the inside of the housing (10) by using the cleaning solution is completed, an enzyme solution containing an enzyme such as trypsin is supplied into the housing (10) through an enzyme solution supply line (not shown) branched from the connection line connecting the pump and the first adapter.

Accordingly, the enzyme solution may chemically separate the cells attached to the cell culture space while passing the cell culture space of the spiral type culture support (40) disposed in the housing.

Here, a process of harvesting the cells cultured while being attached to the cell culture space of the spiral type culture support (40) may be performed by the enzyme solution supplied into the housing in a state in which the spiral type culture support is disposed in the housing. However, the embodiment of the present invention is not limited thereto. The spiral type culture support (40) disposed in the housing may be separated from the housing (10), the first and second culture sheets of the laminated culture sheet or the third and fourth culture sheets of the single culture sheet may be unwound into a flat shape that is a state before being wound, and then the cultured cells attached to the surface of one of the first, second, third, and fourth culture sheets may be separated and harvested using the enzyme solution.

As the cultured cells are separated in a state in which the spiral type culture support is separated from the housing, and the culture sheet of the separated spiral type culture support is unwound into a flat shape that is a state before being wound, the process of separating and harvesting the cells from the culture sheet may be simplified in comparison with a process of separating and harvesting the cultured cells by forcibly injecting and passing the enzyme solution, and the cell harvesting efficiency may be increased.

While the present invention has been particularly shown and described with reference to exemplary embodiments thereof, it will be understood that various changes in form and details may be made therein without departing from the spirit and scope of the following claims.

### DESCRIPTION OF REFERENCE NUMERALS

10: Housing
11: Gripper
12: Invert actuator
20: First cap
21: First adapter
22: First actuator
25: First inflow and outflow hole
30: Second cap
31: Second adapter
32: Second actuator
35: Second inflow and outflow hole
40: Spiral type culture support
41,42: First and second culture sheets
43: Laminated culture sheet
44: Support shaft
110: Pump
120: Bubble trap
130: Culture medium tank
140: Control unit
S1, S2: First and second spaces

1. A spiral type cell culture vessel comprising:
a housing having a certain length with opened both ends;
first and second caps respectively assembled to one end and the other end of the housing; and
at least one spiral type culture support disposed in an inner space of the housing to perform cell culture,
wherein the spiral type culture support comprises at least one culture sheet having one edge that is in contact with and fixed to a support shaft having a certain length, the culture sheet is wound in a spiral shape around the support shaft, a gap formed between an inner side of the spirally wound culture sheet and the culture sheet allows a culture medium to pass in one direction, thereby forming a cell culture space in which the cell culture is performed.

2. The spiral type cell culture vessel of claim 1, wherein the spiral type culture support comprises a laminated culture sheet obtained by vertically laminating two sheets of a first culture sheet having one edge fixed to the support shaft having a certain length and a second culture sheet in which peaks and valleys are repeatedly formed from one end fixed to one edge of one surface of the first culture sheet to the other end fixed to the other edge of the one surface of the first culture sheet, and
the laminated culture sheet that is spirally wound around the support shaft forms a gap between the first culture sheet and the second culture sheet by peaks and valleys of the culture sheet to allow the culture medium to pass in one direction, thereby forming the cell culture space in which the cell culture is performed.

3. The spiral type cell culture vessel of claim 2, wherein the spiral type culture support is formed such that the second culture sheet having overall uniform heights or pitches of the valleys and peaks is disposed between an outer surface and an inner surface, which face each other, of the first culture sheet wound around the support shaft at least two times.

4. The spiral type cell culture vessel of claim 2, wherein the spiral type culture support is formed such that the second culture sheet having overall uniform heights or pitches of the valleys and peaks is disposed between an outer surface and an inner surface, which face each other, of the first culture sheet wound around the support shaft at least two times so that the heights or the pitches of the valleys and peaks are gradually increased in a spiral direction.

5. The spiral type cell culture vessel of claim 2, wherein the second culture sheet has one end that is fixed to one edge of one surface of the first culture sheet by an adhesive and the other end that is fixed to the other edge of the one surface of the first culture sheet by the adhesive.

6. The spiral type cell culture vessel of claim 2, wherein a lower portion of each of the valleys of the second culture sheet, which is in contact with one surface of the first culture sheet, is fixed by an adhesive.

7. The spiral type cell culture vessel of claim 1, wherein the spiral type culture support comprises a single culture sheet made of a third culture sheet which has one edge fixed to the support shaft having a certain length and in which a plurality of linear protrusions are spaced a certain distance from each other on one surface to repeatedly form peaks and valleys, and
the single culture sheet that is wound in a cylinder shape around the support shaft forms a gap between the third culture sheets by the linear protrusions that form the peaks and valleys to allow the culture medium to pass in one direction, thereby forming the cell culture space in which the cell culture is performed.

8. The spiral type cell culture vessel of claim 7, wherein the spiral type culture support is formed such that the linear protrusions having overall uniform heights or pitches is disposed between an outer surface and an inner surface, which face each other, of the third culture sheet wound around the support shaft at least two times.

9. The spiral type cell culture vessel of claim 7, wherein the spiral type culture support is formed such that the linear protrusions each having a height or a pitch that is gradually increased in a spiral direction is disposed between an outer surface and an inner surface, which face each other, of the third culture sheet wound around the support shaft at least two times.

10. The spiral type cell culture vessel of claim 1, wherein the spiral type culture support comprises a single culture sheet made of a fourth culture sheet which has one edge fixed to the support shaft having a certain length and in which a plurality of protruding embossings are spaced a certain distance from each other on one surface, and
the single culture sheet that is wound in a cylinder shape around the support shaft forms a gap between the fourth culture sheets wound by the embossings to allow the culture medium to pass in one direction, thereby forming a cell culture space in which the cell culture is performed.

11. The spiral type cell culture vessel of claim 10, wherein the spiral type culture support is formed such that the embossings having overall uniform heights or pitches is disposed between an outer surface of the fourth culture sheet and an inner surface of the third culture sheet, which are wound around the support shaft at least two times to face each other.

12. The spiral type cell culture vessel of claim 10, wherein the spiral type culture support is formed such that the embossings each having a height or a pitch that is gradually increased in a spiral direction is disposed between an outer surface and an inner surface, which face each other, of the fourth culture sheet wound around the support shaft at least two times.

13. The spiral type cell culture vessel of claim 1, wherein a first space in which the culture medium is temporarily retained and stored is formed between the first cap and one end of the corresponding spiral type culture support.

14. The spiral type cell culture vessel of claim 1, wherein a second space in which the culture medium is temporarily retained and stored is formed between the second cap and the other end of the corresponding spiral type culture support.

15. A cell culture system comprising:
a first actuator configured to operate a first adapter selectively one-to-one corresponding to the first cap or second cap of the housing provided in the spiral type cell culture vessel of claim 1, so that the first adapter is selectively communicated and connected to a first inflow and outflow hole of the first cap or a second inflow and outflow hole of the second cap;
a second actuator configured to operate a second adapter selectively one-to-one corresponding to the first cap or second cap of the housing, so that the second adapter is selectively communicated and connected to the first inflow and outflow hole of the first cap or the second inflow and outflow hole of the second cap; and
a pump disposed on a middle portion of a length of a connection line that is communicated and connected between the first adapter and the second adapter to provide the culture medium stored in a culture medium tank toward the spiral type culture support of the housing and then forcefully circulate the culture medium so that the culture medium discharged from the housing through the cell culture space of the spiral type culture support is returned toward the culture medium tank.

16. The cell culture system of claim 15, further comprising an invert actuator configured to provide an external force to invert the housing by 180° through a gripper configured to clamp the housing.
